(19) United States
(12) Patent Application Publication
(10) Pub. No.: US 2024/0376433 A1
(43) Pub. Date: Nov. 14, 2024

(54) STEM CELL INDUCTION INTO PRECHONDROCYTES AND DIFFERENTIATION INTO CHONDROCYTES BY CIPROFLOXACIN

(71) Applicant: SMART CELL LAB CO., LTD., Incheon (KR)

(72) Inventors: Jung Hwa Kang, Seoul (KR); Sung Hwan Hwang, Seoul (KR); Tae Kyung Ko, Gimpo-si Gyeonggi-do (KR)

(21) Appl. No.: 18/559,810

(22) PCT Filed: Aug. 6, 2021
(86) PCT No.: PCT/KR2021/010376

§ 371 Date: Nov. 9, 2023

(30) Foreign Application Priority Data
May 12, 2021 (KR) 10-2021-0061406

(51) Int. Cl. C12N 5/077; A61K 35/32; A61P 19/04

(52) U.S. Cl. CPC C12N 5/0655; A61K 35/32; A61P 19/04; C12N 2501/10; C12N 2501/11; C12N 2501/113; C12N 2501/165; C12N 2501/999

(57) ABSTRACT

The present invention relates to the effect of ciprofloxacin of inducing stem cells into chondroprogenitor cells or differentiating stem cells into chondrocytes.

A pharmaceutical composition, chondroprogenitor cells, and chondrocytes according to the present invention may be used for preventing or treating cartilage-related diseases.

### TECHNICAL FIELD

The present invention relates to the effect of ciprofloxacin inducing stem cells into chondroprogenitor cells and differentiating stem cells into chondrocytes.

### BACKGROUND ART

Cartilage diseases, which are degenerative diseases, are most frequently caused by the aging of the world's population, so that various studies for the treatment of cartilage diseases have been actively conducted. With aging, in cartilage tissue, the cartilage thickness and the number of chondrocytes decrease, resulting in changes in matrix composition, and changes in cell function are also caused. Further, cartilage is known to be a tissue that cannot regenerate on its own after damage because it uniquely lacks blood vessels, nerves, and lymph tissue.

To treat these cartilage diseases, it is common to use drugs such as analgesics, steroids or non-steroidal anti-inflammatory drugs or supplementary materials such as hyaluronic acid and collagen, which are capable of replenishing cartilage, or to perform artificial joint surgery. However, the above drugs or surgeries have limitations in their effectiveness in maintaining cartilage, or limitations due to factors such as age or allergies.

Therefore, recent attempts have been made to overcome degenerative diseases using stem cells, but efficiency is not high.

In addition, many materials are needed to induce differentiation of stem cells into chondrocytes, and it is difficult to confirm whether stem cells present in the body actually receive help in differentiating into chondrocytes.

Meanwhile, ciprofloxacin is a type of antibiotic that is safe enough to be registered on the WHO Essential Medicines List, and is registered and distributed not only by the FDA but also in South Korea, but the effect of ciprofloxacin inducing cartilage differentiation of stem cells is unknown.

Therefore, the present inventors confirmed that ciprofloxacin can induce differentiation of stem cells into chondrocytes and simultaneously induce stem cells into chondroprogenitor cells (chondrocyte progenitor cell) before maturation into chondrocytes, thereby completing the present invention.

### DISCLOSURE

### Technical Problem

An object of the present invention is to provide a composition for inducing stem cells into chondroprogenitor cells including ciprofloxacin and a growth factor. Another object of the present invention is to provide a composition for inducing differentiation of stem cells into chondrocytes, including ciprofloxacin.

Still another object of the present invention is to provide chondroprogenitor cells induced from a composition for inducing stem cells into chondroprogenitor cells, including ciprofloxacin and a growth factor.

Yet another object of the present invention is to provide chondrocytes differentiated from a composition for inducing differentiation of stem cells into chondrocytes, including ciprofloxacin.

Yet another object of the present invention is to provide a pharmaceutical composition for use in preventing or treating cartilage-related diseases, including a composition for inducing stem cells into chondroprogenitor cells, including ciprofloxacin and a growth factor.

Yet another object of the present invention is to provide a pharmaceutical composition for use in preventing or treating cartilage-related diseases, including a composition for inducing differentiation of stem cells into chondroprogenitor cells, including ciprofloxacin.

Yet another object of the present invention is to provide a pharmaceutical composition for use in preventing or treating cartilage-related diseases, including the chondroprogenitor cells according to the present invention.

Yet another object of the present invention is to provide a pharmaceutical composition for use in preventing or treating cartilage-related diseases, including the chondrocytes according to the present invention.

Yet another object of the present invention is to provide a method for inducing stem cells into chondroprogenitor cells, the method including culturing stem cells in a medium containing a composition for inducing stem cells into chondroprogenitor cells, including ciprofloxacin and a growth factor.

Yet another object of the present invention is to provide a method for treating cartilage-related diseases, the method including administering a composition for inducing stem cells into chondroprogenitor cells, including ciprofloxacin and a growth factor, to a subject in need thereof.

Yet another object of the present invention is to provide a method for treating cartilage-related diseases, the method including administering chondroprogenitor cells induced from a composition for inducing stem cells into chondroprogenitor cells, including ciprofloxacin and a growth factor, to a subject in need thereof.

Yet another object of the present invention is to provide a method for preparing chondroprogenitor cells, the method including culturing stem cells in a medium containing a composition for inducing stem cells into chondroprogenitor cells, including ciprofloxacin and a growth factor.

Yet another object of the present invention is to provide a use of ciprofloxacin and a growth factor for preparing a composition for inducing stem cells into chondroprogenitor cells, including ciprofloxacin and a growth factor.

Yet another object of the present invention is to provide a use of a composition for inducing stem cells into chondroprogenitor cells, including ciprofloxacin and a growth factor, for preparing chondroprogenitor cells.

Yet another object of the present invention is to provide a use of a composition for inducing stem cells into chondroprogenitor cells, including ciprofloxacin and a growth factor, for preparing a pharmaceutical composition for preventing or treating cartilage-related diseases.

Yet another object of the present invention is to provide a use of chondroprogenitor cells induced by a composition for inducing stem cells into chondroprogenitor cells, including ciprofloxacin and a growth factor, for preparing a pharmaceutical composition for preventing or treating cartilage-related diseases.

Yet another object of the present invention is to provide a method for inducing differentiation of stem cells into chondroprogenitor cells, the method including culturing stem cells in a medium containing a composition for inducing differentiation of stem cells into chondroprogenitor cells, including ciprofloxacin.

Yet another object of the present invention is to provide a method for treating cartilage-related diseases, the method including administering a composition for inducing differentiation of stem cells into chondroprogenitor cells, including ciprofloxacin, to a subject in need thereof.

Yet another object of the present invention is to provide a method for treating cartilage-related diseases, the method including administering chondrocytes differentiated from a composition for inducing stem cells into chondrocytes, including ciprofloxacin, to a subject in need thereof.

Yet another object of the present invention is to provide a method for preparing chondrocytes, the method including culturing stem cells in a medium containing a composition for inducing differentiation of stem cells into chondrocytes, including ciprofloxacin.

Yet another object of the present invention is to provide a use of ciprofloxacin for preparing a composition for inducing differentiation of stem cells into chondrocytes.

Yet another object of the present invention is to provide a use of a composition for inducing differentiation of stem cells into chondrocytes, including ciprofloxacin, for preparing chondrocytes.

Yet another object of the present invention is to provide a use of a composition for inducing differentiation of stem cells into chondrocytes, including ciprofloxacin, for preparing a pharmaceutical composition for preventing or treating cartilage-related diseases.

Yet another object of the present invention is to provide a use of chondrocytes induced to differentiate by a composition for inducing differentiation of stem cells into chondrocytes, including ciprofloxacin, for preparing a pharmaceutical composition for preventing or treating cartilage-related diseases.

### Technical Solution

To achieve the above-described objects, the present invention provides a composition for inducing stem cells into chondroprogenitor cells, including ciprofloxacin and a growth factor.

Furthermore, the present invention provides a composition for inducing differentiation of stem cells into chondroprogenitor cells, including ciprofloxacin.

As used herein, the term “stem cells” refers to cells that have the ability to differentiate into cells of various tissues (multi-differentiation potency) by specific differentiation-inducing stimuli along with unlimited proliferation ability (self-renewal capacity) in an undifferentiated state, and may preferably be mesenchymal stem cells.

As used herein, the term “mesenchymal stem cells” refers to cells that have the ability to differentiate into cells of various tissues by specific differentiation-inducing stimuli along with unlimited proliferation ability (self-renewal capacity) in an undifferentiated state. The mesenchymal stem cells of the present invention may be any cells that have the ability to differentiate and proliferate, may be derived from all animals such as humans, monkeys, pigs, horses, cows, sheep, dogs, cats, mice and rabbits, may preferably be derived from humans, more preferably isolated from human adipocyte tissue, and more preferably isolated from human adipose tissue, bone marrow, peripheral blood, umbilical cord blood, or the like, and most preferably derived from body fat.

As used herein, the term “ciprofloxacin (CPFX)” refers to an antibiotic agent used to treat numerous bacterial infections in the related art, and is an antibiotic registered in the WHO Essential Medicines List, which is a list of the most effective and safe drugs essential to the health care system. In the present invention, it was confirmed that when stem cells are cultured in a medium containing such ciprofloxacin, the stem cells are induced into chondroprogenitor cells or the stem cells differentiate into chondrocytes.

As used herein, the term “paracrine effect” refers to the effect that chondroprogenitor cells or chondrocytes do not replace or regenerate chondrocytes or cartilage tissue, but various useful materials secreted from chondroprogenitor cells or chondrocytes can activate chondroprogenitor cells or chondrocytes in vivo to prevent or treat cartilage-related diseases.

In the present invention, the useful material may be SRY-Box Transcription Factor 9 (SOX9) or cartilage oligomeric matrix protein (COMP), but is not limited thereto.

In an exemplary embodiment, as a result of confirming the cytotoxicity of ciprofloxacin by culturing stem cells in a medium, and then administering ciprofloxacin to the medium containing stem cells, since a significant difference in cell viability was not exhibited compared to a control (CONT), it was confirmed that ciprofloxacin exhibits biocompatibility without cytotoxicity.

In an exemplary embodiment, although the composition ratio of the composition of the medium containing ciprofloxacin for inducing stem cells into chondroprogenitor cells according to the present invention is not limited, the basal medium is Dulbecco's Modified Eagle's Medium (DMEM) containing 1 to 10% FBS or an a-MEM medium containing 1 to 10% FBS and may include ciprofloxacin and a growth factor such as EGF, FGF, VEGF, and SGF, and may include preferably EGF, FGF, and the like, but is not limited thereto.

When the EGF is included in a medium composition containing ciprofloxacin for inducing stem cells to chondroprogenitor cells, the EGF may be included in an amount of 0.1 to 100 ng based on the total weight of the medium composition, and when the FGF is included in a medium composition containing ciprofloxacin for inducing stem cells to chondroprogenitor cells, the content of FGF may be 0.1 to 100 ng.

In the present invention, ciprofloxacin for inducing stem cells into chondroprogenitor cells may be included in an amount of 0.01 to 1 μM, preferably 0.1 to 1 μM, based on the total weight of the medium composition, but the content is not limited thereto.

In another exemplary embodiment, although the composition ratio of the composition of the medium containing ciprofloxacin for inducing stem cells into chondroprogenitor cells according to the present invention is not limited, the basal medium is Dulbecco's Modified Eagle's Medium (DMEM) containing 1 to 10% FBS or an a-MEM medium containing 1 to 10% FBS and may include ciprofloxacin.

In the present invention, the medium containing ciprofloxacin for inducing differentiation of stem cells into chondrocytes may not contain other factors such as growth factors in addition to ciprofloxacin.

In the present invention, the ciprofloxacin for inducing stem cells into chondroprogenitor cells may be included in an amount of 0.05 to 10 UM or 0.08 to 8 μM, and may be included in an amount of 0.1 to 6 μM, but the content is not limited thereto.

As used herein, the term “differentiation” refers to a phenomenon in which a structure or function is specialized while cells divide, proliferate, and grow, that is, means that the morphology or function of cells, tissues, and the like of an organism changes in order to carry out the tasks given to them. The differentiation of the present invention refers to the differentiation of stem cells into chondrocytes.

In an exemplary embodiment, it was confirmed that when the stemness marker expression rate, self-renewal ability, and multipotency of chondroprogenitor cells induced from the composition for inducing stem cells to chondroprogenitor cells according to the present invention are compared with those of stem cells, chondroprogenitor cells induced using the composition for inducing stem cells into chondroprogenitor cells, including ciprofloxacin and a growth factor, according to the present invention are capable of self renewal similar to stem cells, can be further induced to differentiate into adipocytes, differentiate into bones and differentiate into cartilage, and express the surface antigen of mesenchymal stem cells.

In another exemplary embodiment, it was confirmed that when the chondrogenic differentiation ability of chondroprogenitor cells induced from the composition for inducing stem cells into chondroprogenitor cells according to the present invention was compared with that of stem cells, it is possible to address problems, such as the cartilage hypertrophy induced by the exponential increase in SOX9 shown when chondrocytes are treated using stem cells and the absence of hyaline cartilage, such as the absence of Col2, which strengthens cartilage such as COMP, when chondroprogenitor cells derived from the composition for inducing stem cells into chondroprogenitor cells, including ciprofloxacin and a growth factor, according to the present invention, are applied to the treatment of chondrocytes.

In still another exemplary embodiment, it was confirmed that when the chondrogenic differentiation ability of chondroprogenitor cells induced from the composition for inducing stem cells into chondroprogenitor cells according to the present invention was compared with that of stem cells, the ability of chondroprogenitor cells to regenerate chondrocytes is better than the ability of stem cells to regenerate chondrocytes when chondrocytes, in which an inflammatory response has been induced, are treated with chondroprogenitor cells induced from the composition for inducing stem cells into chondroprogenitor cells, including ciprofloxacin and a growth factor, according to the present invention.

In yet another exemplary embodiment, it was confirmed that in order to confirm the paracrine effect of chondroprogenitor cells induced from the composition for inducing stem cells into chondroprogenitor cells according to the present invention, when an inflammatory response was induced in chondrocytes using a plate including an insert, and then the insert was treated with chondroprogenitor cells (indirect treatment), the expression levels of ADAMTS4 were reduced by about 35%, and when the corresponding plate was directly treated with the chondroprogenitor cells, the expression levels of ADAMTS4 were reduced by about 76%.

That is, it was confirmed that the chondroprogenitor cells induced from the composition for inducing stem cells into chondroprogenitor cells according to the present invention have an excellent ability to inhibit a cartilage-degrading enzyme.

The cartilage-degrading enzyme is an enzyme that degrade cartilage, and may be an enzyme known as a cartilage-degrading enzyme in the related art, such as ADAMTS4, ADAMTS5, and MMP-13.

Therefore, the present invention provides chondroprogenitor cells induced from a composition for inducing stem cells into chondroprogenitor cells, including ciprofloxacin and a growth factor.

In yet another exemplary embodiment of the present invention, it was confirmed through 2D culture (or two-dimensional culture) that when stem cells were treated with ciprofloxacin, the stem cells were induced to differentiate into chondrocytes. As a result, it was confirmed that cells clustered together as stem cells differentiated into chondrocytes, and that Alcian blue staining, which appears depending on SOX9, was present throughout the chondrocytes.

In yet another exemplary embodiment of the present invention, it was confirmed through 3D culture (or three-dimensional culture) that when stem cells were treated with ciprofloxacin, the stem cells were induced to differentiate into chondrocytes. As a result, it was confirmed that cells clustered together and became tissue-like as stem cells differentiated into chondrocytes, and that Alcian blue staining, which appears depending on SOX9, was present throughout the chondrocytes.

Therefore, the present invention provides chondrocytes differentiated from a composition for inducing differentiation of stem cells into chondrocytes, including ciprofloxacin.

In the present specification, SOX9 is a cartilage differentiation or chondrocyte marker gene or cartilage differentiation marker, such as aggrecan (ACAN), COMP or Col2a. SOX9 is a transcription factor that plays an essential role in cartilage formation and development, and it is known that when SOX9 is excessively suppressed, the expression of ACAN and Col2a, which are the main components of cartilage, is not well expressed (Cha B H et al. Cartilage tissue formation from dedifferentiated chondrocytes by codelivery of BMP-2 and SOX-9 genes encoding bicistronic vector. Cell Transplant 2012).

Therefore, the composition for inducing stem cells into chondroprogenitor cells, including ciprofloxacin and a growth factor, or the composition for inducing differentiation of stem cells into chondrocytes, including ciprofloxacin, according to the present invention may be a composition in which the expression of one or more genes selected from the group consisting of SOX9, ACAN, COMP, and Col2a in the induced cells is increased.

As described above, according to the present invention, chondroprogenitor cells derived from a composition for inducing stem cells into chondroprogenitor cells, including ciprofloxacin and a growth factor, or chondrocytes differentiated from a composition for inducing differentiation of stem cells into chondrocytes, including ciprofloxacin, may be used for preventing or treating diseases requiring cartilage regeneration, such as cartilage-related diseases.

The cartilage-related disease is not limited to, but may be one or more selected from the group consisting of osteoarthritis, arthrosis deformans, dyschondroplasia, degenerative arthritis, rheumatoid arthritis, osteomalacia, osteitis fibrosa, and aplastic bone disease.

Therefore, the present invention provides a pharmaceutical composition for use in preventing or treating cartilage-related diseases, including a composition for inducing stem cells into chondroprogenitor cells, including ciprofloxacin and a growth factor.

In addition, the present invention provides a pharmaceutical composition for use in preventing or treating cartilage-related diseases, including a composition for inducing differentiation of stem cells into chondroprogenitor cells, including ciprofloxacin.

Furthermore, the present invention provides a pharmaceutical composition for use in preventing or treating cartilage-related diseases, including chondroprogenitor cells derived from a composition for inducing stem cells into chondroprogenitor cells, including ciprofloxacin and a growth factor.

Further, the present invention provides a pharmaceutical composition for use in preventing or treating cartilage-related diseases, including chondrocytes differentiated from a composition for inducing differentiation of stem cells into chondrocytes, including ciprofloxacin.

The pharmaceutical composition of the present invention may include a pharmaceutically acceptable carrier, excipient or diluent, in addition to the composition for inducing stem cells into chondroprogenitor cells, including ciprofloxacin and a growth factor, the composition for inducing differentiation of stem cells into chondrocytes, including ciprofloxacin, and the chondroprogenitor cells or chondrocytes according to the present invention, for administration. Examples of the carrier, excipient and diluent include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

Further, the pharmaceutical composition of the present invention can be applied in any dosage form, and more specifically, it may be a formulation for parenteral use. The formulation for parenteral use may be in the form of an injection, an application, a spray, such as an aerosol. More specifically, it may be in the form of an injection.

Examples of a preparation for parenteral administration include an aqueous sterile solution, a non-aqueous solvent, a suspension, an emulsion, a freeze-dried preparation, and a suppository. As the non-aqueous solvent and the suspending agent, it is possible to use propylene glycol, polyethylene glycol, a vegetable oil such as olive oil, an injectable ester such as ethyl oleate, and the like.

In order to formulate the pharmaceutical composition into an injectable formulation, the pharmaceutical composition may be prepared into a solution or suspension by mixing the composition of the present invention with a stabilizer or buffer in water and the solution or suspension may be prepared for ampoule or vial unit administration.

The present invention also provides a method for preventing or treating cartilage-related diseases, the method including: administering the pharmaceutical composition according to the present invention to an individual in need thereof. Specifically, the cartilage-related disease may be one or more selected from the group consisting of osteoarthritis, arthrosis deformans, dyschondroplasia, degenerative arthritis, rheumatoid arthritis, osteomalacia, osteitis fibrosa, and aplastic bone disease, but is not limited thereto.

In the present invention, when the pharmaceutical composition is formulated as a drug, reference may be made to the content disclosed in Remington's Pharmaceutical Sciences, Mack Publishing Company, Easton PA, which is incorporated herein by reference.

In the present specification, “pharmaceutically effective amount” means an amount sufficient to treat diseases at a reasonable benefit/risk ratio applicable to medical treatment, and an effective dosage level may be determined according to factors including the sexually transmitted disease, age, and type of disease of a patient, the severity of disease, the activity of drugs, sensitivity to drugs, administration time, administration route, excretion rate, treatment period, and simultaneously used drugs, and other factors well known in the medical field. The pharmaceutical composition of the present invention may be administered as an individual therapeutic agent or may be administered in combination with other therapeutic agents, and may be administered sequentially or simultaneously with therapeutic agents in the related art.

In addition, the pharmaceutical composition of the present invention may be administered in a single dose or multiple dose. It is important to administer the composition in a minimum amount that can obtain the maximum effect without any side effects, in consideration of all the aforementioned factors, and this amount may be easily determined by those skilled in the art.

The term “individual” of the present invention includes animals such as horses, sheep, pigs, goats, camels, antelopes, and dogs, or humans, which have a cartilage-related disease whose symptoms can be ameliorated by administration of the pharmaceutical composition according to the present invention. By administering the pharmaceutical composition for the prevention or treatment according to the present invention to an individual, cartilage-related diseases may be effectively prevented or treated.

As used herein, “administration” refers to the introduction of a predetermined material to a human or animal by any appropriate method, and for the route of administration of the therapeutic composition according to the present invention, the preventive or therapeutic composition according to the present invention may be orally or parenterally administered via any general route, which may reach a target tissue. Furthermore, the composition for preventing or treating cartilage-related diseases according to the present invention may be administered by any device which may allow an active ingredient to move to a target cell.

A preferred dosage of the pharmaceutical composition according to the present invention varies depending on the condition and body weight of a patient, the degree of a disease, the form of drug, the administration route, and the duration, but may be appropriately selected by a person skilled in the art.

In an exemplary embodiment of the present invention, it was confirmed that stem cells are induced into chondroprogenitor cells and differentiate into chondrocytes in each of a composition for inducing stem cells into chondroprogenitor cells, including ciprofloxacin and a growth factor, or a composition for inducing differentiation of stem cells into chondrocytes, including ciprofloxacin, so that the stem cells may be induced into chondroprogenitor cells using the composition for inducing stem cells into chondroprogenitor cells, including ciprofloxacin and a growth factor, and may be induced to differentiate into chondrocytes using the composition for inducing differentiation of stem cells into chondrocytes, including ciprofloxacin.

Another aspect of the present invention relates to a method for inducing stem cells into chondroprogenitor cells, the method including culturing stem cells in a medium containing a composition for inducing stem cells into chondroprogenitor cells, including ciprofloxacin and a growth factor.

Still another aspect of the present invention relates to a method for treating cartilage-related diseases, the method including administering a composition for inducing stem cells into chondroprogenitor cells, including ciprofloxacin and a growth factor, to a subject in need thereof.

Yet another aspect of the present invention relates to a method for treating cartilage-related diseases, the method including administering chondroprogenitor cells induced from a composition for inducing stem cells into chondroprogenitor cells, including ciprofloxacin and a growth factor, to a subject in need thereof.

Yet another aspect of the present invention relates to a method for preparing chondroprogenitor cells, the method including culturing stem cells in a medium containing a composition for inducing stem cells into chondroprogenitor cells, including ciprofloxacin and a growth factor.

Yet another aspect of the present invention relates to a use of ciprofloxacin and a growth factor for preparing a composition for inducing stem cells into chondroprogenitor cells, including ciprofloxacin and a growth factor.

Yet another aspect of the present invention relates to a use of a composition for inducing stem cells into chondroprogenitor cells, including ciprofloxacin and a growth factor, for preparing chondroprogenitor cells.

Yet another aspect of the present invention relates to a use of a composition for inducing stem cells into chondroprogenitor cells, including ciprofloxacin and a growth factor, for preparing a pharmaceutical composition for preventing or treating cartilage-related diseases.

Yet another aspect of the present invention relates to a use of chondroprogenitor cells induced by a composition for inducing stem cells into chondroprogenitor cells, including ciprofloxacin and a growth factor, for preparing a pharmaceutical composition for preventing or treating cartilage-related diseases.

In the present invention, although the composition ratio of the composition of the medium containing ciprofloxacin for inducing stem cells into chondroprogenitor cells according to the present invention is not limited, the basal medium is Dulbecco's Modified Eagle's Medium (DMEM) containing 1 to 10% FBS or an a-MEM medium containing 1 to 10% FBS and may include ciprofloxacin and a growth factor such as EGF, FGF, VEGF, and SGF, and the growth factor may include preferably EGF, FGF, and the like, but is not limited thereto.

In addition, yet another aspect of the present invention relates to a method for inducing differentiation of stem cells into chondrocytes, the method including culturing stem cells in a medium containing a composition for inducing differentiation of stem cells into chondrocytes, including ciprofloxacin.

Yet another aspect of the present invention relates to a method for treating cartilage-related diseases, the method including administering a composition for inducing differentiation of stem cells into chondrocytes, including ciprofloxacin, to a subject in need thereof.

Yet another aspect of the present invention relates to a method for treating cartilage-related diseases, the method including administering chondrocytes differentiated from a composition for inducing stem cells into chondrocytes, including ciprofloxacin, to a subject in need thereof.

Yet another aspect of the present invention relates to a method for preparing chondrocytes, the method including culturing stem cells in a medium containing a composition for inducing differentiation of stem cells into chondrocytes, including ciprofloxacin.

Yet another aspect of the present invention relates to a use of ciprofloxacin for preparing a composition for inducing differentiation of stem cells into chondrocytes.

Yet another aspect of the present invention relates to a use of a composition for inducing differentiation of stem cells into chondrocytes, including ciprofloxacin, for preparing chondrocytes.

Yet another aspect of the present invention relates to a use of a composition for inducing differentiation of stem cells into chondrocytes, including ciprofloxacin, for preparing a pharmaceutical composition for preventing or treating cartilage-related diseases.

Yet another aspect of the present invention relates to a use of chondrocytes induced to differentiate by a composition for inducing differentiation of stem cells into chondrocytes, including ciprofloxacin, for preparing a pharmaceutical composition for preventing or treating cartilage-related diseases.

In the present invention, “stem cells” refers to cells that have the ability to differentiate into cells of various tissues (multi-differentiation potency) by specific differentiation-inducing stimuli along with unlimited proliferation ability (self-renewal capacity) in an undifferentiated state, and may preferably be mesenchymal stem cells.

In the present invention, the medium including ciprofloxacin and a growth factor used when stem cells are induced into chondroprogenitor cells may include ciprofloxacin in an amount of 0.01 to 1 μM, preferably 0.1 to 1 μM, based on the total weight of the medium composition, and each growth factor may be included in an amount of 0.1 to 100 ng based on the total weight of the medium composition, but the content is not limited thereto.

Furthermore, the medium including ciprofloxacin used when stem cells are induced to differentiate into chondrocytes may include ciprofloxacin in an amount of 0.01 to 10 UM or 0.08 to 8 μM, and ciprofloxacin may be included in an amount of preferably 1 to 6 μM, but the content is not limited thereto.

In the present invention, the medium for culturing stem cells is not limited to, but may be a DMEM or a-MEM medium containing fetal bovine serum (FBS).

The FBS may be contained in an amount of 1 to 15% or 1 to 13%, preferably 1 to 10%, based on the total weight of the medium composition.

As used herein, “prevention” refers to all actions of administering the composition of the present invention to prevent the occurrence of a target symptom or disease, or to delay the occurrence or development thereof.

As used herein, “treatment” refers to all actions of administering the composition of the present invention to ameliorate or eliminate a target symptom or disease.

In the present invention, the contents of ciprofloxacin and the growth factor included in the composition of the present invention may vary as long as they can induce stem cells into chondroprogenitor cells or induce differentiation of stem cells into chondrocytes. When an active ingredient is included in an amount below the lower limit, induction of stem cells into chondrocytes or differentiation of stem cells into chondrocytes may not be exhibited, and when the active ingredient is included in an amount above the upper limit, toxicity may occur in cells.

Numerical values set forth herein should be construed to include a range of equivalents, unless otherwise specified.

### Advantageous Effects

By including ciprofloxacin, the composition according to the present invention can induce stem cells into cartilage or differentiation of stem cells into chondrocytes. In addition, the chondroprogenitor cells according to the present invention exhibit excellent cartilage regeneration effects compared to stem cells.

Therefore, the pharmaceutical composition, chondroprogenitor cells, and chondrocytes according to the present invention can be used for preventing or treating cartilage-related diseases.

### DESCRIPTION OF DRAWINGS

FIG. 1A shows the characteristics of stem cells as the basic characteristics of stem cells.

FIG. 1B shows positive reactions to CD29, 73, 90, and 105 and negative reactions to CD31, 34, and 45 in the immunophenotype expressed by stem cells, as a basic characteristic of stem cells.

FIG. 1C confirmed the ability to differentiate into fat, bone, and cartilage to confirm pluripotency in the differentiation ability of mesenchymal stem cells, as a basic characteristic of stem cells.

FIG. 1D shows the results of showing self-renewal ability through colony forming assay, as a basic characteristics of stem cells.

FIG. 1E shows that the stem cells of the present invention are stable by confirming that there are no changes that would cause genetic defects even after culturing up to 6 passages.

FIG. 2 shows the results of confirming the cytotoxicity of ciprofloxacin.

FIG. 3A shows, using a 2D method, changes in cell characteristics and proteins when ciprofloxacin is used to induce differentiation of mesenchymal stem cells into chondrocytes.

FIG. 3B shows the results of confirming, using a 3D method, changes in cell characteristics and proteins when ciprofloxacin is used to induce differentiation of stem cells into chondrocytes.

FIG. 4A shows the characteristics of chondroprogenitor cells induced by ciprofloxacin.

FIG. 4B shows the karyotyping results of chondroprogenitor cells induced by ciprofloxacin showing there are no genetic mutations in the induced cells.

FIG. 5A shows the results of confirming the expression rates of stemness markers of chondroprogenitor cells and stem cells according to the present invention.

FIG. 5B shows the results of confirming the self-renewal abilities of chondroprogenitor cells and stem cells according to the present invention.

FIG. 5C shows the results of confirming the multipotency (ability to induce fat differentiation, ability to induce osteogenic differentiation, and ability to induce cartilage differentiation) of chondroprogenitor cells and stem cells according to the present invention.

FIG. 6A shows the results of confirming the mRNA expression level of SOX9 according to the induction of the differentiation of chondroprogenitor cells and stem cells according to the present invention.

FIG. 6B shows that in the case of chondroprogenitor cells according to the present invention, COMP increases as SOX9 decreases, but in the case of stem cells, COMP increases at the early stage of differentiation induction and then decreases.

FIG. 7A shows the results of confirming the chondrocyte regeneration effect of chondroprogenitor cells and stem cells according to the present invention.

FIG. 7B shows the results of analyzing the chondrocyte region regenerated in the chondroprogenitor cell treatment group, stem cell treatment group, normal chondrocytes, and inflammatory chondrocytes according to the present invention.

FIG. 8 shows the results of confirming the inhibitory effect of the chondroprogenitor cells according to the present invention on a cartilage-degrading enzyme (ADAMTS4).

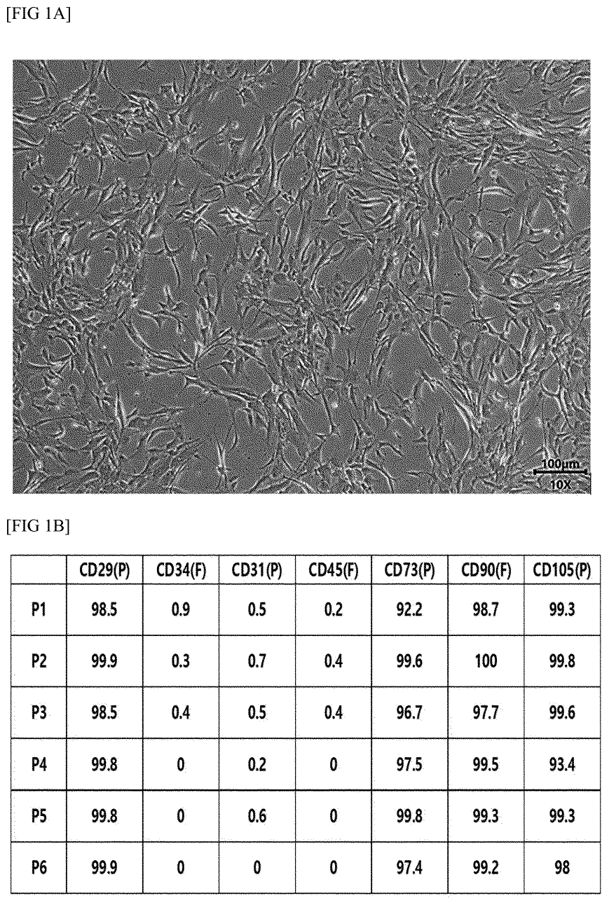

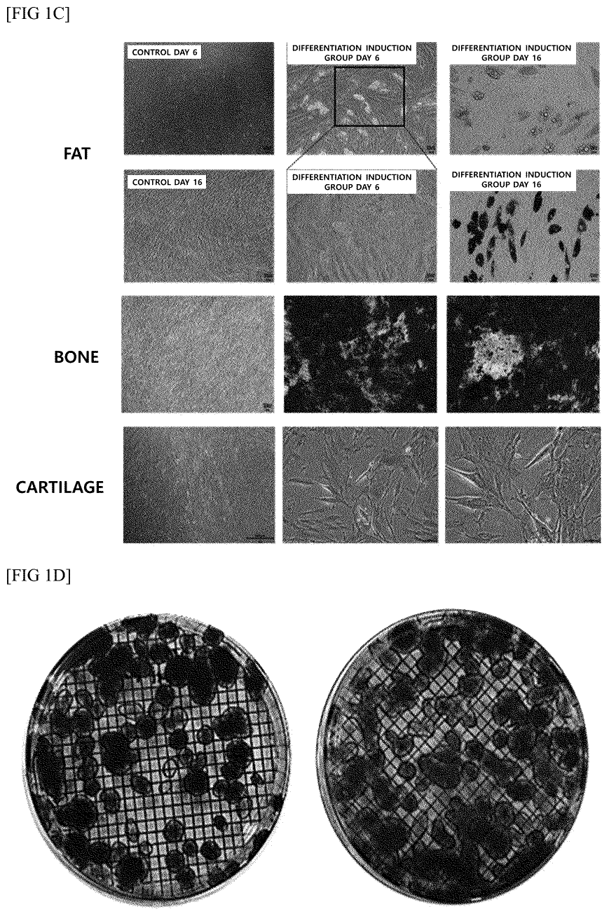

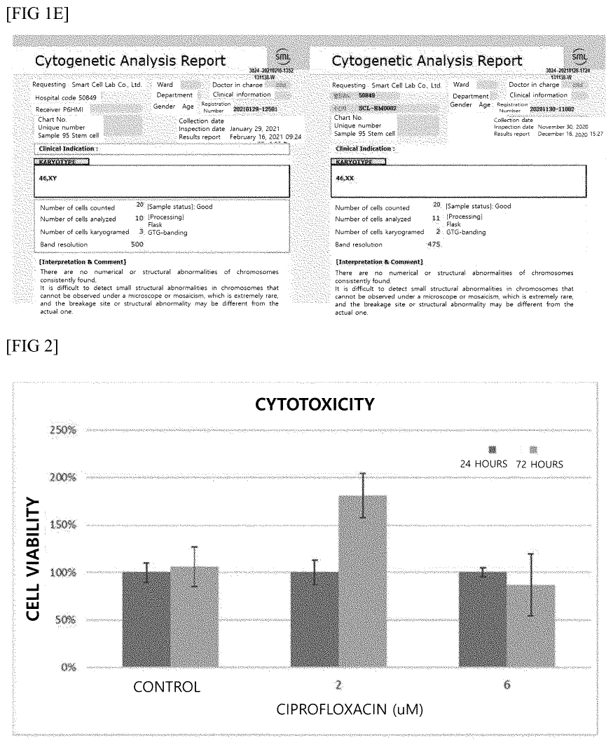

### MODES OF THE INVENTION

The benefits and features of the present invention, and the methods of achieving the benefits and features will become apparent with reference to experimental examples and preparation examples to be described below in detail. However, the present invention is not limited to the experimental examples and the preparation examples to be disclosed below and may be implemented in various other forms, and the present disclosure is provided for rendering the disclosure of the present invention complete and for fully informing those with ordinary skill in the art to which the present invention pertains of the scope of the present invention.

### EXAMPLES

### Example 1. Confirmation of Cytotoxicity of Ciprofloxacin

Stem cells (collected from tissue donated by a hospital for non-profit patient treatment) were cultured by attaching 80% or more of the stem cells to a Dulbecco's modified Eagle's medium (DMEM) or a-MEM containing 1 to 10% FBS.

Thereafter, the cells were collected using Trypsin-EDTA, counted, seeded at 1×10³ cells/well in a 96-well plate, and attached to the plate for 24 hours. Thereafter, ciprofloxacin (Samsung Pharmaceutical, ciprofloxacin injection) was administered according to the concentration, and then cytotoxicity was confirmed for a specific time (up to 166 hours). Next, each well was treated with Cell Counting Kit-8 (hereinafter referred to as CCK, DOJINDO, Cat. No. CK04-13) at 10 μl/well, and then cultured at 37° C. in a 5% CO₂ incubator for 1 hour.

The absorbance was measured using an absorbance meter (Bio-Tek, Epoch2) and quantitatively calculated.

As a result, it was confirmed that ciprofloxacin in stem cells did not exhibit cytotoxicity even when treated up to 6 μM (FIG. 2).

### Example 2. Induction of Differentiation of Stem Cells into Chondrocytes Using Ciprofloxacin (2D)

Stem cells (collected from tissue donated by a hospital for non-profit patient treatment) were cultured by attaching 80% or more of the stem cells to a Dulbecco's modified Eagle's medium (DMEM) or a-MEM containing 1 to 10% FBS.

Thereafter, the cells were collected using Trypsin-EDTA, counted, seeded at 1×10³ cells/well in a 96-well plate, and attached to the plate for 24 hours. Thereafter, ciprofloxacin (Samsung Pharmaceutical, ciprofloxacin injection) was administered according to the concentration, and then cytotoxicity was confirmed for a specific time (up to 166 hours). Next, each well was treated with Cell Counting Kit-8 (hereinafter referred to as CCK, DOJINDO, Cat. No. CK04-13) at 10 μl/well, and then cultured at 37° C. in a 5% CO₂ incubator for 1 hour.

Thereafter, the stem cells induced to differentiate for 16 days were washed with DPBS and fixed with 4% paraformaldehyde for 1 hour.

The completely fixed cells were stained using an Alcian blue solution (Merck, B8438) for 1 hour to 1 day. Thereafter, the cells were washed once with 1 N HCl, and then further washed three times with sterile distilled water.

Finally, the washed sample was observed under a microscope.

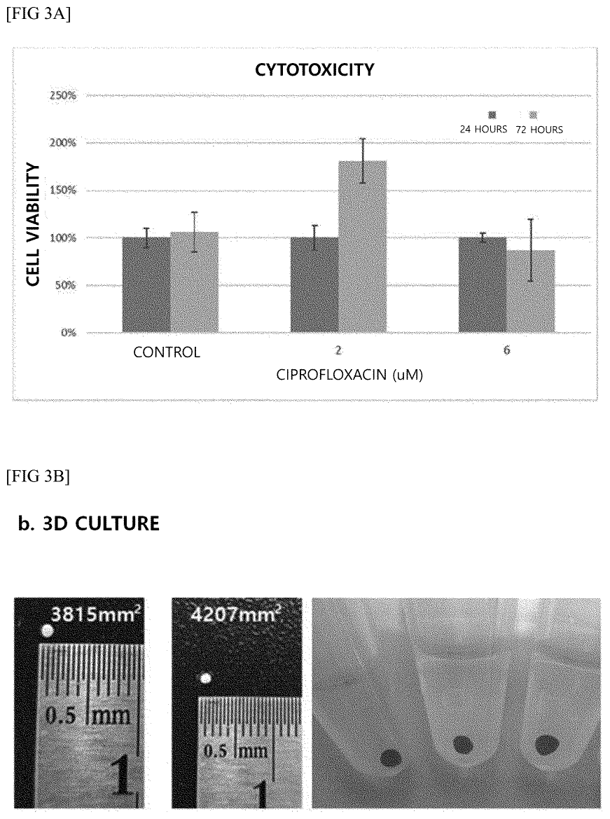

As a result, it was confirmed that cells clustered together as stem cells were induced into chondrocytes, and it could be confirmed that Alcian blue staining, which appears depending on SOX9, was present throughout the chondrocytes (FIG. 3A).

### Example 3. Induction of Differentiation of Stem Cells into Chondrocytes Using Ciprofloxacin (3D)

Stem cells (collected from tissue donated by a hospital for non-profit patient treatment) were cultured by attaching 80% or more of the stem cells to a Dulbecco's modified Eagle's medium (DMEM) or a-MEM containing 1 to 10% FBS.

Thereafter, the cells were collected and counted using Trypsin-EDTA, and 5×10⁵ cells were inoculated into a 15 ml conical tube. A medium containing 0.1 to 6 μM ciprofloxacin was centrifuged to settle the cells at 150 g using a centrifuge (Hanil Science Co., Ltd., Combi415). The cells were cultured in a 5% CO₂ incubator at 37° C. while exchanging the medium every 2 to 3 days.

The stem cells induced to differentiate for 16 days were washed with DPBS and fixed with 4% paraformaldehyde for 1 hour.

The completely fixed cells were stained using an Alcian blue solution (Merck, B8438) for 1 hour to 24 hours. Thereafter, the cells were washed once with 1 N HCl, and then further washed three times with sterile distilled water.

Finally, the washed sample was observed.

As a result, it was confirmed that cells clustered together and became tissue-like as stem cells differentiated into chondrocytes, and it could be confirmed that Alcian blue staining, which appears depending on SOX9, was present throughout the chondrocytes (FIG. 3B).

### Example 4. Induction of Stem Cells into Chondrocytes Using Ciprofloxacin

Stem cells (collected from tissue donated by a hospital for non-profit patient treatment) were cultured by attaching 80% or more of the stem cells to a Dulbecco's modified Eagle's medium (DMEM) or a-MEM containing 1 to 10% FBS. Thereafter, the cells were collected using Trypsin-EDTA and then counted. The cells were suspended in a flask with a medium containing 0.1 to 1 μM ciprofloxacin and a growth factor such as 0.1 to 100 ng EGF and 0.1 to 100 ng FGF, and cultured in a 5% CO₂ incubator at 37° C. while exchanging the medium every 2 to 3 days. This process was repeated for 1 to 3 passages.

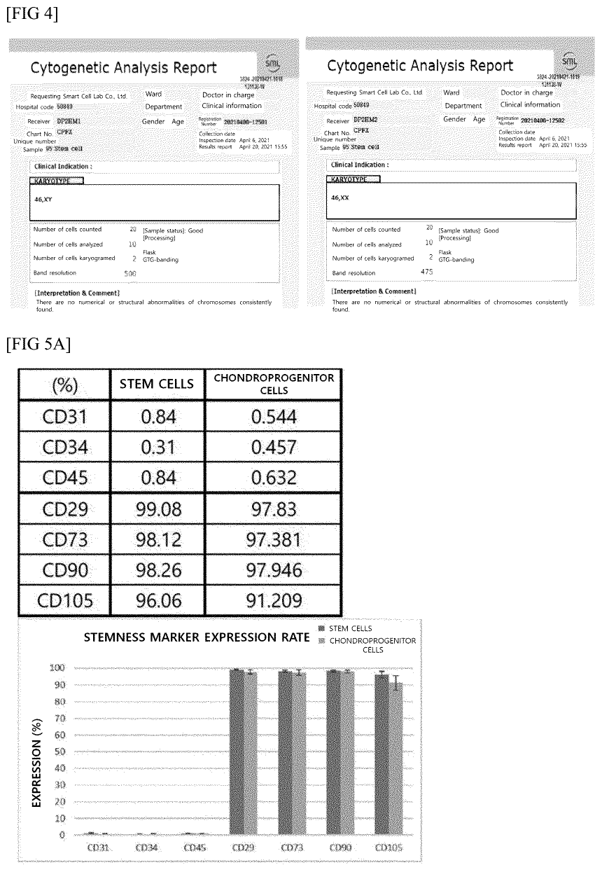

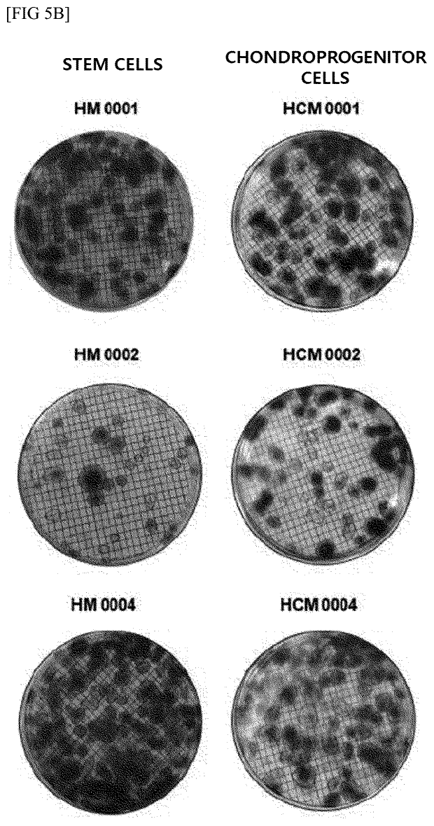

As a result, it was confirmed that stem cells were induced into chondroprogenitor cells from the composition including ciprofloxacin, the cell characteristics of the induced chondroprogenitor cells (FIG. 4A) were confirmed, and it was confirmed that no genetic mutations occurred in the cells by performing karyotyping (FIG. 4B).

### Example 5. Comparison of Chondroprogenitor Cells and Stem Cells

Completely cultured stem cells (collected from tissue donated by a hospital for non-profit patient treatment) and chondroprogenitor cells induced in Example 4 were collected and washed with water.

After the completely washed cells were suspended using a FACS buffer, they were stained for stem cell markers CD73, CD90, CD105, CD31, CD34, and CD45 at 4° C. for 1 hour, and then centrifuged at 1500 RPM for 3 minutes. Thereafter, the cells were washed twice using a FACS buffer, and then expression was confirmed using a FACS (BD, Accuri6) apparatus.

As a result, it was confirmed that a positive marker for stem cells was shown to be positive even in chondroprogenitor cells, and a negative marker for stem cells was shown to be negative even in chondroprogenitor cells.

In addition, it was confirmed that when colony forming assay and bone cell differentiation chondrocyte differentiation ability, and adipocyte differentiation ability were performed using commonly used culture media or kits, the cells are shown to have the same or similar value to that of stem cells.

### Example 6. Comparison of Chondrogenic Differentiation Abilities of Chondroprogenitor Cells and Stem Cells

The chondrogenic differentiation abilities of completely cultured stem cells (collected from tissue donated by a hospital for non-profit patient treatment) and the cartilage progenitor cells prepared in Example 4 above were compared.

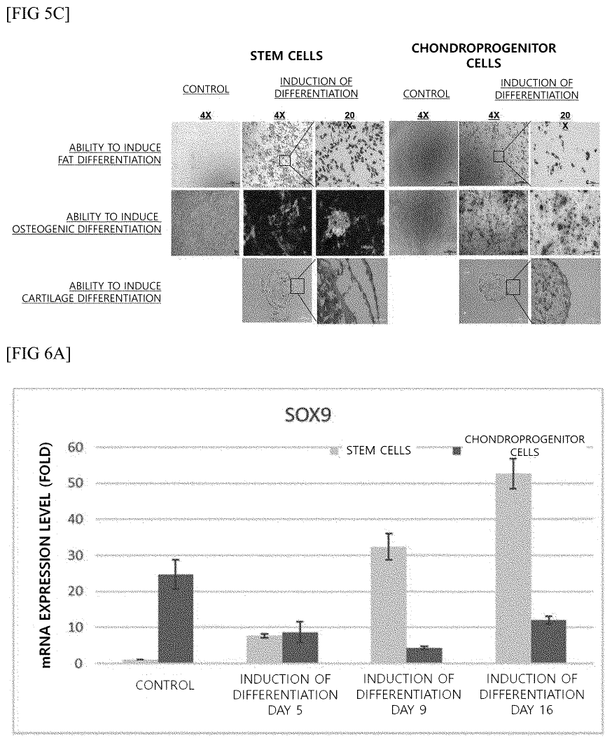

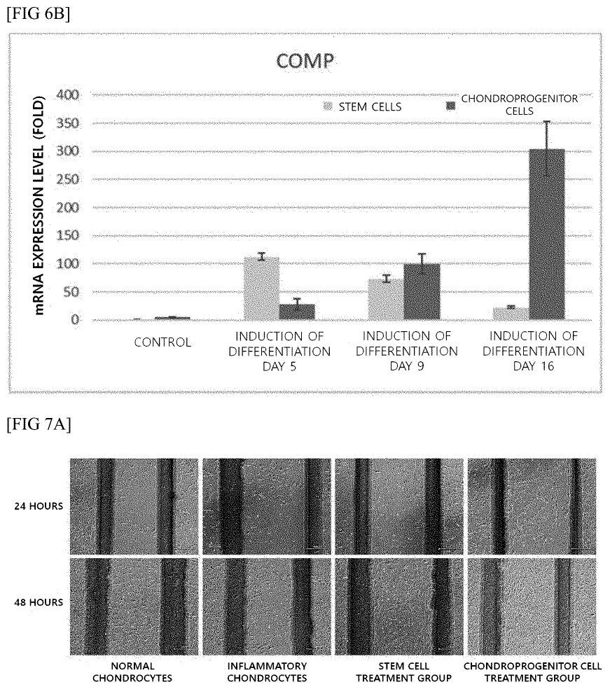

As a result, it was confirmed that in the control, SOX9, an early marker, was basically expressed in chondroprogenitor cells about 20-fold more than in stem cells. However, it was confirmed that as differentiation was induced, the value decreased in chondroprogenitor cells, and only the expression of the early marker continuously increased in stem cells (FIG. 6A).

Thereafter, it was confirmed that COMP, which is a component that constitutes a matrix according to the organization of chondrocytes, increases as SOX9 decreases in chondroprogenitor cells, but in the case of stem cells, COMP increases and then decreases in the early stage of differentiation induction (FIG. 6B).

For this reason, when chondroprogenitor cells are applied to chondrocyte treatment, it is possible to address cartilage hypertrophy (induced by an exponential increase in SOX9) and the absence of hyaline cartilage (the absence of Col2, which hardens cartilage such as COMP), which are problems that occur when chondrocyte treatment is performed using stem cells.

### Example 7. Comparison of Chondrocyte Regeneration Abilities of Chondroprogenitor Cells and Stem Cells

The chondrocyte regeneration ability effects of completely cultured stem cells (collected from tissue donated by a hospital for non-profit patient treatment) and the cartilage progenitor cells prepared in Example 4 above were compared.

An inflammatory response was induced in chondrocytes using LPS. Thereafter, physical stress was induced by applying a scratch of 2 mm or more, and then stem cells or chondroprogenitor cells were treated.

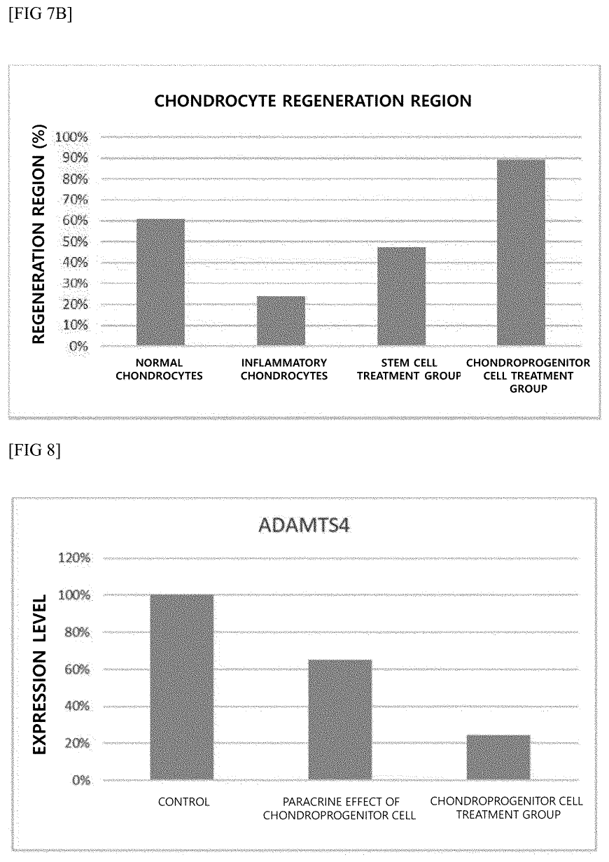

As a result, after an inflammatory response was induced in chondrocytes, some regions were damaged, and when the regions where chondrocytes were regenerated were compared by treating stem cells and chondroprogenitor cells, normal chondrocytes exhibited regeneration of about 61%, but chondrocytes in which inflammation was induced were regenerated by 24%. Furthermore, the group treated with stem cells and chondroprogenitor cells showed a regenerative ability of about 47% or more, and in particular, the group treated with stem cells showed a regenerative ability of about 47% and the group treated with chondroprogenitor cells showed a regenerative ability of about 89% (FIGS. 7A and 7B).

That is, it can be seen that the chondrocyte regeneration ability of chondroprogenitor cells is better than that of stem cells.

### Example 8. Comparison of Cartilage-Degrading Enzyme Inhibitory Effects of Chondroprogenitor Cells

In order to confirm the paracrine effect of chondroprogenitor cells, an inflammatory response was induced in chondrocytes using a plate including an insert (VWR, 734-2719). After the induction, the inhibitory effect on a cartilage-degrading enzyme was confirmed by performing treatment of chondroprogenitor cells on the insert (indirect treatment) or directly treating the corresponding plate with chondroprogenitor cells.

Cartilage-degrading enzymes that induce cartilage degradation include enzymes such as ADAMTS4, ADAMTS5, and MMP-13.

As a result, it was confirmed that the expression levels of ADAMTS4 decreased by about 35% in the case of indirect treatment with chondroprogenitor cells and by about 76% in the case of direct treatment (FIG. 8).

1. A composition for inducing stem cells into chondroprogenitor cells, comprising ciprofloxacin and a growth factor.

2. The composition of claim 1, wherein the stem cells are mesenchymal stem cells.

3. The composition of claim 1, wherein the growth factor is one or more selected from the group consisting of epidermal growth factor (EGF), fibroblast growth factor (FGF), vascular endothelial growth factor (VEGF) and skin growth factor (SGF).

4. A composition for inducing differentiation of stem cells into chondrocytes, comprising ciprofloxacin.

5. The composition of claim 4, wherein the stem cells are mesenchymal stem cells.

6.-44. (canceled)

45. A method for treating cartilage-related diseases, the method comprising administering a chondroprogenitor cells to a subject in need thereof, wherein the chondroprogenitor cells are induced from a composition for inducing stem cells into chondroprogenitor cells, comprising ciprofloxacin and a growth factor.

46. The method of claim 45, wherein the stem cells are mesenchymal stem cells.

47. The method of claim 45, wherein the growth factor is one or more selected from the group consisting of epidermal growth factor (EGF), fibroblast growth factor (FGF), vascular endothelial growth factor (VEGF) and skin growth factor (SGF).

48. A method for treating cartilage-related diseases, the method comprising administering chondrocytes to a subject in need thereof, wherein the chondrocytes are induced from a composition for inducing differentiation of stem cells into chondrocytes, comprising ciprofloxacin.
